(19) United States
(12) Patent Application Publication
(10) Pub. No.: US 2007/0141122 A1
(43) Pub. Date: Jun. 21, 2007

(54) Nutritional composition and method of manufacture

(75) Inventor: Ken Reulein, Encinitas, CA (US)

Correspondence Address: MITCHELL P. BROOK;C/O LUCE, FORWARD, HAMILTON & SCRIPPS LLP, 11988 EL CAMINO REAL, SUITE 200, SAN DIEGO, CA, 92130, US

(73) Assignee: Angel Sports Nutrition, Inc.

(21) Appl. No.: 11/315,429

(22) Filed: Dec. 21, 2005

(51) Int. Cl. A61K 36/254; A61K 36/899; A61K 47/00; A61K 33/26; A61K 31/522

(52) U.S. Cl. 424/439; 424/728; 424/646; 424/750; 514/263.31

(57) ABSTRACT

A nutritional composition, and method of making it, that includes a carbohydrate, an insulin potentiator, an amino acid, a muscle growth stimulator, an energy increaser and at least one flavoring ingredient. The composition may be provided in dry form for addition to a fluid or in liquid forms. The composition is ingested to improve endurance and muscle tissue repair after exercise. In an embodiment the composition includes vanadyl sulfate, gamma oryzanol and American ginseng.

### FIELD OF THE INVENTION

The present invention relates to a nutritional composition such as provided in nutrition, sports and energy drinks.

### BACKGROUND OF THE INVENTION

Athletes, both amateur and professional, often engage in strenuous activities, which subject their bodies to enormous stresses and fatigue. In such activities, the body consumes energy and other nutritional components in muscle and other metabolic or physical functions. In additions such activities can have an affect of damaged muscle tissue and dehydration. Recovery from such muscle damage and dehydration might involve rehydration or rest over a period of time. Sometimes it is often desired to take part in another activity before a full recovery time has elapsed.

Various compositions are known for providing nutrients for sports, fitness or other nutritional purposes. Many known compositions provide various mixtures of carbohydrates, sugars and proteins or particular amino acids. Others add other ingredients such as fats. One example in a published patent is provided in Strahl U.S. Pat. No. 6,039,987, which relates to a composition intended to reduce dehydration, supply energy and prevent cramps. The composition includes electrolytes, carbohydrates and quinine and may be provided in aqueous or dry forms. Electrolytes are intended to replace the essential ions lost during perspiration. Carbohydrates are included as a combination of fructose and glucose. Quanine is included to prevent muscle cramping.

Another example is rehydration drink such as described in Simone U.S. Pat. No. 5,397,786. The drink is intended to reduce vertigo/dizziness, lightheadedness, fatigue and muscle cramps caused by dehydration. Ingredients of the drink include a carbohydrate, an electrolyte, an energy enhancer, an antioxidant, a membrane stabilizer and at least one neuromuscular function enhancer. A further example of a sports drink is “Gatorade” (a registered trademark of Stokely-Van Camp Inc. of Chicago, Ill.), which provides a product line of sports drinks. Those sports drinks are advertised as providing carbohydrates to provide energy during exercise and electrolytes for replacing those lost during perspiration.

There also are numerous other health supplements that provide various combinations of carbohydrates, amino acids and energy increasers designed to increase endurance, prevent dehydration and to provide necessary building blocks for muscle tissue repair and recovery.

There are various deficiencies in the known nutritional supplements in that they may address some types of bodily energy, repair or rehydration requirements, but may not address a full range of such requirements. Accordingly, there exists a need for a nutritional composition, powder or drink that provides ingredients directed towards mechanisms for improved muscle repair and recovery, improved hydration, and improved delivery of energy or amino acid constituent delivery to muscles during exercise. There is a further need for nutritional supplements providing combinations that are effective while being appropriate for athletes living with diabetes.

### SUMMARY OF THE INVENTION

The present invention alleviates to a great extent the disadvantages of known sports and energy drinks by providing a nutritional composition including ingredients that, in combination, are provided to enhance energy and amino acid delivery to muscles, and otherwise improve endurance and muscle tissue repair. In one embodiment the composition includes a carbohydrate, an insulin potentiator, an amino acid, a muscle growth stimulator, an energy increaser and flavoring. In an example, the composition includes vanadyl sulfate as an insulin potentiator, gamma oryzanol as a muscle growth stimulator and American ginseng as an energy increaser.

The composition may be provided in dry or liquid forms. For example, the composition may be supplied as a powder or tablet configured to be mixed with a fluid, such as water, for ingestion. Alternatively, the composition may be provided as a fluid in either concentrated for mixture with a fluid, or ready-to-drink strength, for example as a bottled sports or energy drink. The composition may also be packaged in bulk or as single servings.

These and other features and advantages of the present invention will be appreciated from review of the following detailed description of the invention.

### DETAILED DESCRIPTION OF THE PREFERRED EMBODIMENTS

In the following paragraphs, the present invention will be described in detail by way of example with reference to the attached drawings. Throughout this description, the preferred embodiments and examples shown should be considered as exemplars, rather than as limitations on the present invention. As used herein, the “present invention” refers to any one of the embodiments of the invention described herein, and any equivalents. Furthermore, reference to various feature(s) of the “present invention” throughout this document does not mean that all claimed embodiments or methods must include the referenced feature(s). The “nutritional composition” and “sports drink” of the present invention refer to a composition of ingredients that may be provided in liquid or dry forms. For example, the liquid form may be an aqueous mixture that may be concentrated or ready-to- ingest. Furthermore, the dry form may include loose powder, or compressed forms that may be added to and partially or completely dissolved and/or held in suspension. For example, a dry form may be a loose powder that is mixed in water for ingestion.

Broadly speaking, the nutritional composition comprises a combination or mixture of all or a subset of components from the following categories of substances: 1) carbohydrates, 2) insulin potentiators, 3) amino acids, 4) muscle growth stimulators, and 5) energy increasers. Each of the categories will be described in greater detail below. In addition, a composition according to the present invention may further include additional active or inactive ingredients, sweeteners, natural and/or artificial flavoring and/or coloring agents.

It is understood that the cells in the human body acquire energy through the metabolism of carbohydrates, including sugars. During and after strenuous exercise the body's natural stores of carbohydrates become depleted. Ingestion of carbohydrates during and after exercise help to assure that the natural stores do not become dangerously low and also help to recover and return the body to normal carbohydrate levels.

The nutritional composition of the present invention includes at least one carbohydrate. Preferably, the carbohydrate is easily metabolized so that it provides a rapidly absorbed energy source, which allows the natural carbohydrate levels to be efficiently restored and/or maintained. Examples of carbohydrates suitable for use in the nutritional composition include sugars, such as sucrose, fructose and/or dextrose, although any form of carbohydrate can be used. In one embodiment the dextrose is used in the nutritional composition. Dextrose is a monosaccharide that occurs naturally in food. Dextrose also has a high glycemic index, which indicates that it has a high ability to raise blood glucose levels when it is digested.

The body relies upon insulin to allow cells to metabolize glucose by increasing the permeability of the cell wall, thereby allowing glucose to pass into the cells. A sudden increase of carbohydrate may require the body to increase production of insulin. As used herein, the term “insulin potentiator” refers to a substance that increases the production or effectiveness of insulin in the body. The composition of the present invention includes an insulin potentiator in order to with the production and/or transmission of insulin so as to promote metabolization of glucose, providing energy delivery. A further benefit of providing an insulin potentiator is that athletes or other individuals having diabetes may not be able to increase production of insulin as desired. An example of an insulin potentiator, which may be used in the present invention, is vanadyl sulfate. Vanadyl sulfate has been used to improve insulin sensitivity and to reduce blood sugar level in people living with diabetes. When vanadyl sulfate is ingested, insulin levels within the body are understood to increase which enables the body to take advantage of the increase in carbohydrates provided by the nutritional composition, such as an increase in blood sugar levels. As a result, the increase in insulin (or enhanced delivery) associated with the vanadyl sulfate increases the access of the components of the nutritional composition to enter the muscle cells thereby providing the user with the benefits of the various components, one of which is the increased ability to metabolize carbohydrates. The nutritional composition of the present invention may include 15-60 mg of vanadyl sulfate. In one embodiment, the composition provides approximately 30 mg of vanadyl sulfate are provided per serving, although any desired amount of vanydyl sulfate or other insulin potentiator may be provided.

The increased cell wall permeability attendant with insulin also allows amino acids to be utilized by cells. Amino acids are the basic building blocks of proteins, which form muscle tissue. It is desirable to increase the exposure of muscle cells to amino acids so that damaged muscle tissue may be repaired and additional muscle tissue may be formed. At least one amino acid is included in the nutritional composition of the present invention so that the increased permeability of the cell walls may be utilized so that the rate of muscle tissue repair may be improved. A single amino acid or a combination of various amino acids or proteins may be included in the nutritional composition.

An example of an amino acid that may be included in the nutritional composition is arginine which may be provided in the form of diarginine malate. In one embodiment, the nutritional composition of the present invention may include 1-10 g of diarginine malate, although any amount of diarginine malate or other amino acid provider may be used. Preferably, 3 g of diarginine malate is included. It is also believed that diarginine malate becomes more effective when it is combined with additional ingredients, i.e., it provides a synergistic effect.

Another example of an amino acid that may be included in the nutritional composition is creatine. Creatine in general is a compound that is naturally synthesized by the liver, pancreas and kidneys. Creatine may act as an energy buffer which allows for the quick conversion of adenosine diphosphate (ADP) into adenosine triphosphate (ATP) for short durations. Creatine supplements are understood to increase the amount of creatine stored in their muscle tissue to provide an increased capacity for converting ADP into ATP which in turn provides an increase in energy availability. Such an increase in energy is desirable so that more strenuous activities may be undertaken with less threat of fatigue. Additionally, creatine has been found to increase body mass by allowing growth of muscle, which may be desirable for some athletes, such as bodybuilders.

Creatine may be provided in a number of different forms such as, for example, creatine monohydrate, creatine ethyl ester, dicreatine malate and tricreatine malate. Preferably, creatine is provided in the form of dicreatine malate in the present invention because it has been shown to provide a higher rate of user responsiveness, although other forms may be provided instead or in addition to dicreatine malate.

The nutritional composition of the present invention may include 5-10 grams of creatine, preferably in the form of dicreatine malate. Furthermore, the nutritional composition may be offered with differing quantities of creatine so that an athlete may follow any desired supplementation schedule.

Another example of an amino acid suitable for the nutritional composition of the present invention is taurine. Taurine assists in the transport of various substances to the muscle tissue, such as other amino acids. In addition, it is known to play a role in digestion and is believed to be an important agent in counteracting congestive heart failure, cystic fibrosis, diabetes and epilepsy. A serving of the nutritional composition may include 0-5 g of taurine. Preferably, a single serving provides approximately 2 g of taurine.

A further example of a suitable amino acid is glutamine which may be provided in the form of N-Acetyl glutamine. Glutamine is an essential building block for protein and muscle tissue. It has generally been recommended following traumatic injury in order to reduce healing time. A serving of the nutritional composition may include 0-5 g of N-Acetyl glutamine. Preferably, a single serving provides approximately 2 g of the amino acid.

In addition to the amino acids described above, other ingredients may be included in the nutritional composition of the present invention that stimulate the growth of muscles. For example, gamma oryzanol which may promote the growth of muscle tissue by stimulating the release of growth hormones in the body may be added to the composition. That muscle tissue growth results in more efficient muscle tissue repair. Gamma oryzanol is a lipid that may be found in rice bran oil. In addition to the muscle growth stimulation effect, gamma oryzanol may be used to reduce fat deposits in the arteries and for its antioxidant properties. The dosage of gamma oryzanol per serving may be 500-1000 mg.

Guanidinopropionic acid is another exemplary muscle growth stimulator. The substance is also believed to help regulate the function of insulin, which as described above, may allow the body to construct new muscle tissue more efficiently. Such a function is ideal to start the repair of muscle tissue that has been damaged through strenuous activity. The composition of the present invention may include approximately 0-2 g of guanidinopropionic acid. Preferably, the composition includes 500 mg of guanidinopropionic acid per serving.

Glycocyamine may also be used in the nutritional composition to stimulate muscle growth especially for users that are not responsive to creatine. One explanation for such users'inability to utilize creatine is that their livers are highly efficient at breaking down the creatine, thereby preventing it from reaching the muscle tissue. Glycocyamine, or guanidinoacetic acid, is a creatine precursor that is converted into creatine by the liver. As a result, the efficiency of those creatine non-responders'livers may be utilized to provide increased natural creatine to their muscle tissue. Glycocyamine may be included in a quantity ranging from approximately 0 to 3 g per serving. Preferably, approximately 500 mg of glycocyamine is provided.

Betaine may also be added to improve the effectiveness of glycocyamine. In particular, betaine may reduce levels of homocysteine, which may be harmful to blood vessels, that is created during the conversion of glycocyamine into creatine. Studies also suggest that betaine may help protect the liver against fatty deposits, which may occur from protein malnutrition or poorly controlled diabetes. Preferably, the concentration of betaine in the nutritional composition is higher than the concentration of glycocyamine. In an embodiment, betaine may be provided in an amount that is approximately four (4) times the amount of glycocyamine. In a preferred embodiment, 2 g of betaine and 500 mg of glycocyamine are provided in a single serving of a nutritional composition.

One or more energy increasers may be added to the nutritional composition to provide the user with a quick burst of energy. American ginseng is an energy increaser and it is widely used to strengthen the immune system and increase endurance and strength while reducing reaction time. The substance acts as a stimulant and increases metabolism. The addition of American ginseng provides an athlete with a boost of energy during exercise and/or during the recovery period to counteract fatigue. American ginseng is available in water, water and alcohol, alcohol liquid extracts and powders. The nutritional composition of the present invention may provide 1-2 g of American ginseng per serving.

Another energy increaser that may be used in the nutritional composition is caffeine. Caffeine is a well-known stimulant that may be included to provide quick energy to improve endurance and to avoid the onset of fatigue. Caffeine is readily available as would be appreciated by a person having ordinary skill in the art. In a preferred embodiment, the composition includes approximately 300 mg of caffeine, however, it should be appreciated that the amount of caffeine may be 100-300 mg.

Although the carbohydrate may provide sweetening in addition to an energy source, it may be desirable to add a non-sugar sweetener that does not increase the quantity of carbohydrates in the nutritional composition. Any form of non-sugar sweetener can be used in quantities depending on a level of sweetness desired. Among the examples of know sweeteners are acesulfame potassium, aspartame, saccharine etc. Acesulfame potassium is calorie free and approximately 200 times sweeter than sugar. The substance is also widely used in food products around the world. Acesulfame potassium is neither metabolized nor stored in the body, instead it is rapidly excreted from the body unchanged. Additionally, the American Diabetes Association has indicated that the sweetener is appropriate for people living with diabetes. The quantity of acesulfame potassium may be varied according to taste depending on the level of sweetness desired.

Sucralose is another example of a non-sugar sweetener that may be utilized. Sucralose is a low-calorie sweetener that is derived from sugar and is approximately 600 items sweeter than sugar. Similar to acesulfame potassium, sucrose is not metabolized by the body and is excreted unchanged. As a result, it neither provides carbohydrates nor calories. Sucralose is also used in many products worldwide. The quantity of sucralose may be varied according to taste depending on the level of sweetness desired.

Citric acid may be included to improve the flavor of the nutritional composition. The substance is a weak organic acid that is commonly found in citrus fruits. Citric acid is a well-known flavoring and preservative in food and beverages and it is readily available as would be appreciated by a person having ordinary skill in the art. The amount of citric acid may be selected according to the preferred flavor or acidity desired.

The nutritional composition may also include various natural and artificial flavors and coloring agents to improve the taste and appearance. Any suitable flavoring materials or coloring agents may be used as would be appreciated by a person having ordinary skill in the art. In addition, the composition may have any desired flavor, for example it may be provided in flavors such as fruit punch, lemonade or another fruit or fruit medley flavor. In addition, the composition may be added to any type of tea, which in addition may be sweetened or fruit flavored.

### EXAMPLE

A formula for a nutritional composition according to an embodiment of the present invention is provided as an illustration below. The following constituents are combined to form the nutritional composition. It should be understood that the amounts shown, are provided to illustrate proportionality in an exemplary single serving and not to limit the batch size. In the embodiment shown, the single serving size is approximately 30 g, however it should be appreciated that a single serving is not limited to that quantity. Furthermore, the example provided is directed to a powdered form nutritional composition in accordance with the present invention.
12 g sugar
3 g diarginine malate
3 g dicreatine malate
2 g taurine
2 g n-acetyl glutamine
2 g betaine (anhydrous)
1 g gamma oryzanol
1 g American ginseng
500 mg guanidinopropionic acid
500 mg glycocyamine
300 mg caffeine
30 mg vanadyl sulfate
non-sugar sweetener
fruit punch flavoring
   Such a powdered composition may be added to approximately 10-12 oz of water to create a ready-to-drink composition that is fruit punch flavored.

It shall be appreciated that the nutritional composition of the present invention may be provided in bulk or single servings. For example, a quantity of a powdered form of the composition sufficient for multiple single servings may be provided to a user so that it may be apportioned into individual servings by the user. In one example, 750 g of the powdered composition may be provided in a single container, thereby providing approximately twenty-five (25) single servings, with each serving being 30 g. In another embodiment, a package containing a single serving of the composition is provided, which may increase the transportability of the composition. For example, an athlete may desire to carry a single serving with them, for example in a gym bag, so that the composition may be more readily available for ingestion at the time of, or shortly after, exercising.

The nutritional composition of the present embodiment may be manufactured by mixing dry forms of the desired components. Alternatively, the nutritional composition may be manufactured by mixing aqueous solutions of the desired components, or by mixing a combination of dry and aqueous forms of the various constituents.

Thus, it is seen that a nutritional composition providing improved endurance and muscle repair and recovery is provided. One skilled in the art will appreciate that the present invention can be practiced by other than the above-described embodiments, which are presented in this description for purposes of illustration and not of limitation. The description and examples set forth in this specification and associated drawings only set forth embodiment(s) of the present invention. The specification is not intended to limit the exclusionary scope of this patent document. Many designs other than the above-described embodiments will fall within the literal and/or legal scope of the following claims, and the present invention is limited only by the claims that follow. It is noted that various equivalents for the particular embodiments discussed in this description may practice the invention as well.

1. A nutritional composition for ingestion comprising:
a carbohydrate;
a non-carbohydrate insulin potentiator;
an amino acid;
a muscle growth stimulator;
at least one energy increaser;
at least one of guanidinopropionic acid, glycocyamine and di-arginine malate; and
at least one flavoring ingredient;
wherein the composition is provided in dry form.

2. The nutritional composition of claim 1 wherein the carbohydrate is dextrose.

3. The nutritional composition of claim 1 wherein the insulin potentiator is vanadyl sulfate.

4. The nutritional composition of claim 1 wherein the amino acid is provided as at least one of di-arginine malate, di-creatine malate, taurine and n-acetyl glutamine.

5. The nutritional composition of claim 1 wherein the muscle growth stimulator is gamma oryzanol.

6. (Canceled)

7. The nutritional composition of claim 1 wherein the energy increaser is one of American ginseng and caffeine.

8. The nutritional composition of claim 1 wherein the flavoring ingredient is citric acid.

9. The nutritional composition of claim 1 further comprising a non-sugar sweetener.

10. The nutritional composition of claim 9 wherein the non-sugar sweetener is acesulfame potassium.

11. The nutritional composition of claim 1 wherein the muscle growth stimulator is gamma oryzanol and the insulin potentiator is vanadyl sulfate.

12. The nutritional composition of claim 1 wherein the muscle growth stimulator is gamma oryzanol and the energy increaser is American ginseng (Panax quinguefolius).

13. The nutritional composition of claim 1 wherein the insulin potentiator is vanadyl sulfate and the energy increaser is American ginseng.

14. The nutritional composition of claim 11 wherein the energy increaser is American ginseng (Panax quinquefolius).

15. A nutritional composition for ingestion comprising:
gamma oryzanol;
American ginseng (Panax quinguefolius);
vanadyl sulfate;
at least one of guanidinopropionic acid, glycocyamine and di-arginine malate; and
at least one carbohydrate;
wherein the composition is provided in dry form.

16. (Canceled)

17. (Canceled)

18. The nutritional composition of claim 15 wherein the dry form is a powder.

19. The nutritional composition of claim 15 wherein the dry form is a tablet.

20. An aqueous sports drink wherein a 10-12 ounce serving comprises:
between about 10 g and 20 g of dextrose;
between about 500 mg and 1000 mg of gamma oryzanol;
between about 15 mg and 60 mg of vanadyl sulfate; and
between about 1 g and 2 g of American ginseng (Panax quinquefolius).

21. The aqueous sports drink of claim 20 further comprising between 5 g and 10 g of di-creatine malate.
